(19) United States
(12) Patent Application Publication
(10) Pub. No.: US 2019/0217290 A1
(43) Pub. Date: Jul. 18, 2019

(54) Encoded Chip Based Micro-Array, Preparation Method Thereof and Application

(71) Applicant: Suzhou Institute of Nano-Tech and Nano-Bionics (Sinano), Chinese Academy of Sciences, Suzhou (CN)

(72) Inventors: Jiong LI, Suzhou (CN); Kexiao ZHENG, Suzhou (CN)

(73) Assignee: Suzhou Institute of Nano-Tech and Nano-Bionics (Sinano), Chinese Academy of Sciences, Suzhou (CN)

(21) Appl. No.: 16/307,021

(22) PCT Filed: Sep. 27, 2017
(86) PCT No.: PCT/CN2017/103694

§ 371 Date: Dec. 4, 2018

(30) Foreign Application Priority Data
Sep. 28, 2016 (CN) 201610860592.3

(51) Int. Cl. B01L 3/00; G01N 21/64; G01N 21/76; C12Q 1/68

(52) U.S. Cl. CPC B01L 3/502707; G01N 21/64; B01L 2300/0636; C12Q 1/68; G01N 21/76

(57) ABSTRACT

The disclosure discloses an encoded chip based micro-array and a preparation method thereof. In a typical embodiment, the preparation method comprises: providing a carrier, which has at least one fluid accommodating cavity, wherein at least one carrying surface is distributed in the fluid accommodating cavity; uniformly coating the carrying surface with an adhesive; adding a selected fluid to the fluid accommodating cavity till the carrying surface is immersed by the selected fluid; depositing encoded microchips dispersed in the selected fluid on the carrying surface, and enabling the encoded microchips to be combined with the adhesive distributed on the carrying surface; and curing the adhesive, thereby fixing the microchips onto the carrying surface. The disclosure further discloses a biochemical kit based on the micro-array, a biochemical detection system and method, and the like. Compared with the prior art, the disclosure shows outstanding advantages in the aspects of multiplicity, efficiency, sensitivity and the like of detection, high-throughput and high-precision sample detection can be achieved, operations are simple, and preparation is simple, convenient and economical.

### TECHNICAL FIELD

The present disclosure specifically relates to an encoded chip based micro-array, a preparation method thereof and application, such as application in multiple biochemical detection and analysis.

### BACKGROUND

Biochip technology is a multidisciplinary technology emerged in the late twentieth century. Depending on engineering technologies including microelectronics, micromechanics, opto-mechatronics and the like, the biochip technology uses one chip to integrate discontinuous procedures, including sample preparation, chemical reaction, analysis and detection and the like, in the life science research so as to achieve continuation, integration and microminiaturization of the processing procedure.

The traditional planar micro-array chips are prepared by mainly utilizing an in-situ synthesis method and a spotting method. However, such preparation scheme will generate many problems, for example, sensing materials will be contaminated with each other in the preparation procedure, the density is low by the spotting method, and the costs are over-high by the in-situ synthesis method.

Suspension array chip technology is also known as micro-carrier technology and is a recently developed new chip technology. It carries out multi-target detection and analysis on the fluid mainly through specificity interaction between sensitive sensing materials fixed on encoded micro-particles and to-be-detected samples. Compared with the traditional planar micro-array chip technology, the suspension array chip technology has many outstanding advantages such as higher yield, more flexible detection target arrangement, faster reaction, higher quality of experimental results and the like.

The current graph encoded micro-carrier suspension array chip carries out detection by the flow cytometry in order to avoid problems that micro-carriers are hard to focus to obtain signals in a motion state in the solution and are easy to be mutually interfered due to a blockage problem in the detection procedure, but as a result, such chip cannot carry out on-chip detection, and the detection throughput is largely limited, and meanwhile, detections on graph encoded micro-carriers by the flow cytometry also generate a problem that graphs are hard to be recognized during movement.

### SUMMARY

A main objective of the present disclosure is to provide an encoded chip based micro-array, a preparation method thereof and application, in order to overcome the disadvantages in the prior art.

Embodiments of the present disclosure provide a preparation method of encoded chip based micro-array, which comprises:

providing at least one carrying surface;

uniformly coating the carrying surface with an adhesive;

depositing a plurality of encoded microchips on the carrying surface in a discrete state, and enabling the plurality of encoded microchips to be combined with the adhesive distributed on the carrying surface; and

curing the adhesive, thereby fixing the encoded microchips onto the carrying surface.

In some preferred embodiments, the preparation method of encoded chip based micro-array comprises:

providing a carrier, wherein the carrier has at least one fluid accommodating cavity, and at least one carrying surface is distributed in the fluid accommodating cavity;

uniformly coating the carrying surface with an adhesive;

adding a selected fluid to the fluid accommodating cavity till the carrying surface is immersed by the selected fluid;

depositing encoded microchips (which may also be called as microchips for short below) dispersed in the selected fluid on the carrying surface, and enabling the encoded microchips to be combined with the adhesive distributed on the carrying surface; and

curing the adhesive, thereby fixing the encoded microchips onto the carrying surface.

In some embodiments, the preparation method comprises: depositing the encoded microchips dispersed in the selected fluid on the carrying surface under the action of any one or at least two components selected from a group of a gravity field, an external magnetic field and an external electric field, and enabling the encoded microchips to be combined with the adhesive distributed on the carrying surface.

In some preferable embodiments, the carrying surface is a flat surface.

In some preferable embodiments, the encoded microchips are distributed on the carrying surface in a lying state.

Embodiments of the present disclosure further provide an encoded chip based micro-array, which comprises a carrier and a plurality of encoded microchips, wherein the carrier has at least one carrying surface, and the plurality of encoded microchips are distributed on the carrying surface in a discrete state and are adhered and fixed to the carrying surface.

In some preferred embodiments, the encoded chip based micro-array comprises a carrier and a plurality of encoded microchips, wherein the carrier has at least one fluid accommodating cavity, at least one carrying surface is distributed in the fluid accommodating cavity, and the plurality of encoded microchips are distributed on the carrying surface in a discrete state and are adhered and fixed to the carrying surface.

In some preferable embodiments, the encoded microchips are fixed to and combined with the carrying surface through the cured adhesive coating the carrying surface.

In some preferable embodiments, the carrying surface is a flat surface.

In some preferable embodiments, the encoded microchips are distributed on the carrying surface in a lying state.

In some preferable embodiments, the carrying surface is the bottom end surface of the fluid accommodating cavity.

Embodiments of the present disclosure further provide application of the encoded chip based micro-array.

Furthermore, embodiments of the present disclosure provide a biochemical kit, which comprises any one encoded chip based micro-array described above.

Furthermore, the biochemical kit comprises a dataset, and the dataset comprises locating information and decoding information corresponding to the plurality of encoded microchips distributed on the carrying surface.

Furthermore, embodiments of the present disclosure provide a biochemical detection system, which comprises:

any one biochemical kit described above; and

an optical imaging device, which is at least used for collecting associated image information when the biochemical kit detects a to-be-detected analyte.

Furthermore, the biochemical detection system comprises a data processing device, which is at least used for processing the image information collected by the optical imaging device and combining the dataset in the biochemical kit to achieve qualitative and/or quantitative detection on the to-be-detected analyte.

Furthermore, embodiments of the present disclosure provide a biochemical detection method, which comprises:

providing any one biochemical kit described above;

adding a liquid phase sample possibly including the target substance to the fluid accommodating cavity of the micro-array, and ensuring that the target substance is combined with the capture substance fixed to the carrier; and then

by an imaging device, collecting image information of the micro-array, and comparing the collected image information with the dataset in the biochemical kit so as to achieve qualitative and/or quantitative detection on the to-be-detected analyte.

Furthermore, the image information collected by the imaging device comprises at least one of image coding information, optical strength information and spectrum coding information, which are shown by each encoded microchip after each encoded microchip and the to-be-detected analyte generate reactions.

Compared with the prior art, the present disclosure shows outstanding advantages in the aspects of multiplicity, efficiency, sensitivity and the like of detection by randomly dispersing and fixing the plurality of encoded microchips on the surface of the substrate in order to form the micro-array and then detecting the encoded microchips through the imaging technology; moreover, operations are simple, preparation costs are low, and high-throughput and high-precision sample detection can be achieved.

### BRIEF DESCRIPTION OF THE DRAWINGS

To describe the technical schemes in the embodiments of the present disclosure or the prior art more clearly, the following briefly introduces the accompanying drawings required for describing the embodiments or the prior art. Apparently, the accompanying drawings in the following description show merely some embodiments of the present disclosure, and a person of ordinary skill in the art may still derive other drawings from these accompanying drawings without creative efforts.

FIG. 1 is a schematic diagram of a preparation method and application of an encoded chip based micro-array in a typical embodiment of the present disclosure.

FIG. 2 is an image of a micro-fluidic channel under a microscope in embodiment 4 of the present disclosure.

FIG. 3 is a schematic diagram of an encoded chip fixed in the fluidic channel in embodiment 4 of the present disclosure.

FIG. 4 is a schematic diagram of a plurality of encoded chips fixed in the fluidic channel in embodiment 4 of the present disclosure.

FIG. 5 and FIG. 6 respectively are a light field image and a fluorescence image of the micro-fluidic channel after reaction in embodiment 5 of the present disclosure.

### DESCRIPTION OF THE EMBODIMENTS

In view of deficiencies of the prior art, the inventors of the present disclosure provide the technical scheme of the present disclosure based on long-range research and a lot of practices. The following further explains and describes the technical scheme, implementation processes and principles of the present disclosure.

In the present specification, the terms “include,” “comprise,” or any other variant is intended to cover a non-exclusive inclusion, so that procedures, methods, objects or devices that include a series of elements not only include those elements, but also include other elements that are not explicitly listed, or further include elements inherent to the procedures, the methods, the objects or the devices.

In one aspect, embodiments of the present disclosure provide a preparation method of encoded chip based micro-array, which comprises:

providing a carrier, wherein the carrier has at least one fluid accommodating cavity, and at least one carrying surface is distributed in the fluid accommodating cavity;

uniformly coating the carrying surface with an adhesive;

adding a selected fluid to the fluid accommodating cavity till the carrying surface is immersed by the selected fluid;

depositing encoded microchips dispersed in the selected fluid on the carrying surface, and enabling the encoded microchips to be combined with the adhesive distributed on the carrying surface; and

curing the adhesive, thereby fixing the encoded microchips onto the carrying surface.

Wherein at least the following requirement should be met after the adhesive is cured: the encoded microchips do not fall off from the carrying surface and do not move along with the fluid when the fluid is added to the fluid accommodating cavity of the carrier, undergoes operations of oscillation, ultrasonic, agitation and the like, and is heated (the heating temperature of the fluid does not exceed the decomposition temperature and the melting temperature of a material for curing the adhesive).

The above encoded microchips may also be called as suspension arrays or liquid arrays, and their technical principles may refer to the following document: J. Immunol. Methods., 2000, 243, 243-255; Exp. Hematol., 2002, 30, 1227-1237.

An encoded microchip applicable to the present disclosure may have the following characteristics:

a). the size (such as length, width, diameter and the like) of the encoded microchip is from 1 micron to 1000 microns;

b). the encoded microchip utilizes solid materials (which may be made from inorganic nonmetallic materials such as silicon, ferric oxide and the like, metal materials, fluorescent materials, high molecular materials and the like), and it may show magnetism, electrical property and the like; and

c). the encoded microchip has a designed optical structure (such as outline shape, size, spectrum, surface pattern and the like or a combination thereof), the optical structure may be used for identifying the identity of the encoded microchip (namely carrying out optical coding including, but not limited to, graph coding, spectrum coding and the like), and such appearance structure (and the encoded microchip per se) may be imaged by an imaging device (which may be a visible-light imaging device or a fluorescence imaging device, and which may also be a microscopic optical imaging device and the like), and recognized by the human eyes or a machine (namely decoded).

Wherein the above encoded microchip has no limitations in shape, and it may be spherical, sheet-shaped and block-shaped, or in other regular or irregular shapes.

In some typical embodiments, a type of encoded microchips comprises a substrate with an optical identification code. The substrate may be spherical, sheet-shaped and block-shaped, or in other regular or irregular shapes. The optical identification code comprises a graph identification code or a spectrum code, preferably the graph identification code, wherein the graph identification code may be identified by the human eyes, a visible-light optical device or an invisible-light optical device, and the spectrum code may be identified in a manner of fluorescence-emission spectroscopic imaging and the like.

In some typical embodiments, a type of encoded microchips may comprise a substrate and a microstructure that is connected with the substrate and is used as an optical identification code, wherein the microstructure may be formed on the substrate in an integrated processing manner, and may also be combined with the substrate in a manner of physical or chemical deposition, chemical growth, adhesion, metallic bonding and the like. The microstructure may be a graph structure, and it may be processed and formed on the surface of the substrate, may also be formed by hollowing the substrate, or may also be formed by hollowing parts of the substrate and filling the hollowed parts with a specific optical substance (which has a special light refractive index and a high reflective rate or can emit lights with a specific wavelength).

In some more specific embodiments, a type of encoded microchips comprises a transparent substrate and an opaque planar microstructure as the graph identification code, and the opaque planar microstructure is distributed on the surface of the transparent substrate.

More specifically, the design scheme of the type of encoded microchips may comprise: growing and/or processing a group of planar microstructures on a solid substrate substantially transmissive to visible light via a micromachining technique to be as the graph identification code, wherein the group of planar microstructures is formed by a specific material (preferably a silicon material such as silicon oxide, silicon nitride and the like) and has the high reflectivity corresponding to visible lights with a specific waveband. After undergoing optical imaging (via a microscope) under the illumination of the visible light, the graph identification code may be identified by the human eyes or a computer-controlled sensor, and may be transformed into a digital code (such as a barcode or a two-dimensional code) according to a preset rule or program in order to indicate the identity (including the ID and the type) of the encoded microchip. Therefore, a large amount of different types of microchips may be encoded by processing different graphs. The preparation flow of some typical encoded microchips may refer to Chinese Patent Publication Number CN101543755A and CN102788779A. Furthermore, by taking the Chinese Patent Publication Number CN102788779A for example, an encoded graph in a microchip has the high reflectivity to visible lights with a specific wavelength based on the dielectric high-reflective film principle, so it also has very low light transmittance and very high optical contrast ratio in comparison with a substrate made of a transparent material. When an optical device is used for imaging the microchip, the obtained image has a bright substrate and a dark encoded graph, so the human eyes or identification software may be very easy to recognize its graph structure and decode it.

In addition, other types of encoded microchips applicable to the present disclosure may be further selected from, but not limited to, industry-known various fluorescent encoded micro-particles (such as dye fluorescent encoded micro-particles, quantum dot fluorescent encoded micro-particles and the like), SERS encoded micro-particles, magnetic suspension encoded microchips, and the like.

Furthermore, the surface of each encoded microchip may be fixed with a specific biochemical reagent (which may also be a capture agent such as antibody, antigen, protein, enzyme, enzyme substrate, nucleic acid and the like, and which may specifically capture a target substance such as chemical molecules, biological molecules and the like) by utilizing a method (such as a chemical coupling method, a physical absorption method and the like).

More specifically, in some embodiments, to a group of to-be-detected biochemical analytes (such as antigen, nucleic acid and the like), each detection index of the analytes is distributed with a specific graph code (each graph code and each detection index are in one-to-one correspondence and are not repeated with each other), and then an agent (such as antibody, nucleic acid DNA/RNA and the like) capable of specifically capturing the analytes is coupled and fixed onto the surface of a microchip with the corresponding graph code by utilizing a biochemical method, so the modified microchip may specifically capture the analyte, corresponding to its graph code, onto the surface, wherein the coupling reaction generally generates in a solution, and after the reaction is complete, each functionalized microchip may be cleaned and recycled, so that the recycled microchips may be mixed according to multiple detection demands.

In the present disclosure, the above adhesive may be a special fluid, wherein the fluid may be arranged on the carrying surface in a coating and the like manner so as to form a thickness-controllable coating or may be injected to fill a certain stereo space, and it also has the capability of adhering more than two objects. Especially, after coating the carrying surface, the adhesive in the present disclosure may be self-leveling under the action of its gravity and cover the whole carrying surface. Additionally, the adhesive may be cured under certain outside conditions (for example, adding a curing agent, prolonging the reaction time, heating and the like) and then loses its original fluidity. Adhesives applicable to the present disclosure may be any industry-known proper types of adhesives, including, but not limited to, an epoxy type of adhesives, a silica gel type of adhesives and the like.

The above carrying surface may be distributed at any proper positions in the fluid accommodating cavity of the carrier, such as the bottom end surface of the fluid accommodating cavity, a side wall thereof, or a center fixed to the fluid accommodating cavity by one or more supporting bodies.

Additionally, the above carrying surface may be any forms of planes or curved surfaces, and it should be beneficial to the imaging device to image each encoded microchip distributed on the carrying surface as accurate as possible.

Preferably, the carrying surface is a flat surface.

Certainly, according to actual application demands, a bulge and/or recess structure may also be arranged on some parts of the carrying surface on the premise that the imaging of each encoded microchip in the imaging device should not be influenced.

In some preferable embodiments, the encoded microchips are distributed on the carrying surface in a lying state, that is, a planar micro-array is formed on the carrying surface.

In some embodiments, the preparation method may specifically comprise:

uniformly dispersing a plurality of encoded microchips in a solvent, thereby forming an encoded microchip suspension liquid as the selected fluid, wherein the solvent comprises water and/or an organic solvent, preferably water or an aqueous solution such as a conventional buffer solution (such as PBS and the like typically);

adding the encoded microchip suspension liquid to the fluid accommodating cavity till the carrying surface is immersed by the encoded microchip suspension liquid;

depositing the plurality of encoded microchips in the encoded microchip suspension liquid on the carrying surface in a discrete state, and enabling the plurality of encoded microchips to be combined with the adhesive distributed on the carrying surface; and

curing the adhesive, thereby fixing the plurality of encoded microchips onto the carrying surface and then forming an encoded microchip based micro-array.

In some embodiments, the preparation method may also specifically comprise:

adding a selected fluid to the fluid accommodating cavity till the carrying surface is immersed by the selected fluid including water and/or an organic solvent, preferably water or a buffer solution;

dispersing the plurality of encoded microchips in the selected fluid, depositing the plurality of encoded microchips on the carrying surface in a discrete state, and enabling the plurality of encoded microchips to be combined with the adhesive distributed on the carrying surface; and

curing the adhesive, thereby fixing the plurality of encoded microchips onto the carrying surface and then forming an encoded microchip based micro-array.

In some embodiments, the preparation method may further comprise: depositing the encoded microchips dispersed in the selected fluid on the carrying surface under the action of any one or at least two components selected from a group of a gravity field, an external magnetic field and an external electric field, and enabling the encoded microchips to be combined with the adhesive distributed on the carrying surface.

In the embodiments, the distribution density of the encoded microchips on the carrying surface may be regulated by regulating the flow state of the selected fluid in the fluid accommodating cavity, the strength of the magnetic field, the strength of the electric field and the like, or regulating a combining form of the gravity field, the magnetic field and the electric field, thereby reducing even avoiding crossing and piling conditions of the encoded microchips on the carrying surface.

In some preferable embodiment, the preparation method may further comprise: depositing the encoded microchips dispersed in the selected fluid on the carrying surface under its own weight, and enabling the encoded microchips to be combined with the adhesive distributed on the carrying surface, wherein the density of the encoded microchips is greater than the density of the selected fluid.

In the embodiments, the sedimentation velocity and the sedimentation state of the encoded microchips may be regulated by carrying out a manner of oscillation, ultrasonic, agitation and the like on the selected fluid in the fluid accommodating cavity in order to enable the selected fluid to generate perturbation, so the distribution density of the encoded microchips on the carrying surface is regulated.

Furthermore, in the embodiments, the fluid in the fluid accommodating cavity may generate perturbation by carrying out a manner of oscillation, ultrasonic, agitation and the like after the adhesive is cured, so that microchips which are not firmly combined with the carrying surface may fall off from the carrying surface; and the fall-off microchips may be removed from the fluid accommodating cavity with the fluid together; so the micro-array formed by the encoded microchips is fixed in the fluid accommodating cavity.

In some preferable embodiments, the carrying surface is a horizontal carrying surface. Preferably the encoded microchips are distributed on the carrying surface in the lying state, thereby forming the planar micro-array.

In some specific embodiments, the preparation method may also comprise: uniformly coating the carrying surface with a fluidic adhesive.

In some preferable embodiment, the preparation method comprises: applying the fluidic adhesive to the carrying surface so that the adhesive naturally spreads out to form a plane on the carrying surface.

In some preferable embodiment, the encoded microchips may be selected from silicon-based encoded microchips, and the adhesive is selected from silicone adhesive. As silicon materials, the microchips have relatively stronger combining force with silicone such as polydimethylsiloxane (PDMS), and when the silicone is gradually cured due to the polymerization reaction, the microchips are firmly combined with the surface of the silicone.

Certainly, the above adhesive may also be selected from conventional adhesives such as epoxy resin and the like.

Preferably, the carrying surface is the bottom end surface of the fluid accommodating cavity.

The above carrier may be various materials and forms, preferably a container with a flat bottom, and more preferably, but not limited to, a multi-well plate, a micro-plate, a micro-fluid based device (such as a micro-fluidic chip), a container similar to a watch glass and the like, which are widely applied to biochemical detection and clinic diagnosis.

In some specific embodiment, a few amount of macromolecular adhesive (preferably the silicone such as PDMS) precursor mixtures may be added to the bottom of the fluid accommodating cavity of the above carrier (generally such as a micro-plate and the like). At this time, the adhesive is fluidic and will naturally spread out to form a plane on the bottom of the container under the action of the gravity. Before the adhesive is not cured, a mixed suspension liquid of the encoded microchips corresponding to the whole to-be-detected analyte is added to the container, so that the microchips deposit on the surface of the silicone and disperse at the whole bottom of the fluid accommodating cavity of the carrier When the silicone is gradually cured due to the polymerization reaction, the microchips are firmly combined with the bottom of the fluid accommodating cavity of the carrier. Therefore, a planar micro-array formed by a group of dispersed microchips may be formed at the bottom of the fluid accommodating cavity, wherein the basic unit of the planar micro-array is a microchip that does not generate displacement along with the movement of fluid in the container. After the curing of the adhesive is complete, the fluid in the fluid accommodating cavity may be removed.

Furthermore, the preparation method may further comprise: imaging and identifying the plurality of encoded microchips distributed on the carrying surface so as to locate and decode each encoded microchip and obtain a dataset including locating information and decoding information corresponding to the plurality of encoded microchips distributed on the carrying surface, wherein the decoding information may include, but is not limited to, graph coding information shown by the encoded microchips, spectrum coding information shown by one or more emitted lights in a specific wavelength range, optical strength information and the like.

In the above embodiments, the utilized imaging detection manner may be selected from various luminescence imaging detection manners, including, but not limited to, fluorescence, chemiluminescence and bioluminescence and the like.

In some specific embodiment, an imaging device such as a microscopic optical device and the like may be used for collecting images of a microchip array in each fluid accommodating cavity under the condition of light field illumination, and its definition and focus position ensure that all encoded graphs on the surfaces of the microchips should be clearly shot. By the image processing software, the whole images of the microchip array in each fluid accommodating cavity are combined to form one image (which may be named as image B), and the image comprises position and coding information of all microchips in each fluid accommodating cavity. Then, all of the microchips in the image are located and decoded by using the image identifying and decoding software. Finally, the information (including, but not limited to, coordinates, outline, codes and the like) of the whole microchips in each fluid accommodating cavity is recorded in a computer and is saved as an e-file (which is the dataset).

In the other aspect, embodiments of the present disclosure correspondingly provide an encoded chip based micro-array, which comprises a carrier and a plurality of encoded microchips, wherein the carrier has at least one fluid accommodating cavity, at least one carrying surface is distributed in the fluid accommodating cavity, and the plurality of encoded microchips are distributed on the carrying surface in a discrete and are adhered and fixed to the carrying surface.

Furthermore, the encoded microchips are fixed to and combined with the carrying surface through a cured adhesive cured coating the carrying surface.

Wherein the forms, the distribution positions and the like of the carrying surface, the material selection range of the carrier, the structures, the forms, the materials, the types and the like of the encoded microchip, and the materials, the thickness, the arrangement forms and the like of the adhesive all may be described in the foregoing and are not described again herein.

In the other aspect, embodiments of the present disclosure further provide a biochemical kit, which comprises any one encoded chip based micro-array described above.

Furthermore, the biochemical kit further comprises a dataset, wherein the dataset comprises locating information and decoding information corresponding to the plurality of encoded microchips distributed on the carrying surface.

Furthermore, the biochemical kit may further comprise an operating instruction and the like.

Furthermore, the biochemical kit may further include, but is not limited to, at least one of a buffer solution, a detection agent, diluent and lotion.

A typical detection agent includes, but is not limited to, at least one component selected from a group of a detection antibody or ligand with a fluorescent dye label, a detection antibody or ligand with a biotin label, an avidin-fluorescent protein (preferable phycoerythrin) conjugate, an avidin-horseradish peroxidase compound and a chemiluminiscence agent (such as luminol, luminol derivative, acridinium ester, luciferase, and an oxidizing agent such as hydrogen peroxide and the like).

In the other aspect, embodiments of the present disclosure further provide a biochemical detection system, which comprises:

any one biochemical kit described above; and

an optical imaging device, which at least is used for collecting associated image information when the biochemical kit detects a to-be-detected analyte.

Furthermore, the biochemical detection system may further comprise: a data processing device (which may be selected from a personal computer system and the like), wherein the data processing device at least is used for processing the image information collected by the optical imaging device and combines the dataset in the biochemical kit to achieve qualitative and/or quantitative detection of the to-be-detected analyte.

Furthermore, the image information collected by the optical imaging device includes, but is not limited to, at least one of image coding information, optical strength information and spectrum coding information, which are shown by each encoded microchip after each encoded microchip and the to-be-detected analyte generate reactions.

In the other aspect, embodiments of the present disclosure further provide a biochemical detection method, which comprises:

providing any one biochemical kit described above;

adding a liquid phase sample possibly including the target substance to the fluid accommodating cavity of the micro-array, and ensuring that the target substance is combined with the capture substance fixed to the carrier; and

by an imaging device, collecting image information of the micro-array, and comparing the collected image information with the dataset in the biochemical kit so as to achieve qualitative and/or quantitative detection on the to-be-detected analyte.

Furthermore, the image information collected by the imaging device comprises at least one of image coding information, optical strength information and spectrum coding information, which are shown by each encoded microchip after each encoded microchip and the to-be-detected analyte generate reactions.

In some embodiments, the biochemical detection method may further comprise: a liquid phase sample possibly including the target substance to the fluid accommodating cavity of the micro-array, ensuring that the target substance is combined with the capture substance fixed to the carrier, and forming a certain optical signal (such as fluorescence and spectrum) on the surface of the microchip, in which the target substance is captured, by generating reactions with other agents in the kit. In general, the strength of the optical signal is positively correlated with the consistency of the target substance.

In some embodiments, the biochemical detection method may further comprise: by the imaging device, collecting image information of the micro-array, and comparing the collected image information with the dataset in the biochemical kit so as to identify the identity information of each chip, and then extracting optical signal strength of each chip in the image in combination with detection results of a standard substance, thereby achieving all applications such as qualitative, semi-quantitative and quantitative detections on the analyte.

Referring to FIG. 1, in some specific embodiment of the present disclosure, regularly arranged planar micro-arrays are prepared without various precise and expensive mechanical spotting devices or in-situ synthesis devices, and only depending on natural sedimentation of the microchips under the action of gravity, position allocation of different detection points may be achieved; each microchip is located and decoded by the following imaging and image identifying operations, and users may handle the coordinates of each detection point on the micro-array and the identity of the detected analyte in advance; therefore, an application mode completely equivalent to the traditional planar micro-array is achieved.

Furthermore, the to-be-detected analyte undergoes a bioluminescence imaging method so as to obtain its detection signals, wherein a chemiluminiscence imaging method or a fluorescence imaging method and the like. In an application scenario (namely a client such as a clinical laboratory or a scientific research laboratory), there is low resolution requirements on the imaging device as long as the outline of the chip may be clearly shot, so that the extract requirements on the luminescence/fluorescence signal strength may be met. In comparison with the e-file, which is provided with the kit at the same time, analyte information corresponding to each microchip may be obtained. Image identifying and decoding procedures are omitted in the client detection procedure, so that the detection time is largely saved.

By taking a sandwich chemiluminiscence immunodetection for an example, an application method of the micro-array and the kit provided by the present disclosure comprises:

(1) a sample (liquid sample) is added to the fluid accommodating cavity of the carrier to culture, wherein a capture agent (an antibody) pre-coupled on the surface of each microchip will generate specific combination with the corresponding analyte (antigen) in the sample; and after the sample is washed off, various detection agents (generally a detection antibody with a biotin label, an avidin-horseradish peroxidase (SA-HRP) compound, a chemiluminiscence agent and the like) are sequentially added in order to generate reactions;

(2) on the surface of the microchip capturing the analyte, a sandwich compound is formed and the SA-HRP is combined, so a chemiluminiscence reaction may be catalyzed, and then optical signals generate on the surface of the microchip; at this time, the bottoms of the fluid accommodating cavities of the carriers placed in a dark room are shot by the chemiluminiscence imaging device, and all view pictures of the bottom of the fluid accommodating cavity of each carrier are combined into one image, wherein the obtained image (image L) has the dark background, the brightness shown by the microchip generating the chemiluminiscence reaction is remarkably greater than the brightness of the background on the obtained image, and the consistency of the analyte is positively correlated with the light intensity; and

(3) overlapping comparison is carried out on the image L and the image B by using software, and data of the e-file (namely the dataset) is imported, so positions, outlines and codes of all microchips at the bottom of the fluid accommodating cavity of each carrier may be labeled on the image L, wherein the code of each microchip represents the identity of the analyte corresponding to the microchip, and the strength value of the optical signal inside the outline of the microchip represents the consistency value of the analyte; and a standard curve is established by using standard substances with known consistency, so the analyte with an unknown consistency may be quantified, wherein the bottom of the fluid accommodating cavity of each carrier has a plurality of different encoded microchips, so the consistency quantified information of a plurality of analytes may be obtained via once detection.

The application form may be expanded according to requirements. For example, the avidin-horseradish peroxidase (SA-HRP) may be replaced with fluorescence-labeled avidin (such as phycoerythrin-avidin and SA-RPE) and the like, and the chemiluminiscence imaging device may be replaced with fluorescence imaging device and the like, that is, the sandwich chemiluminiscence immunodetection may be changed into fluorescence immunodetection.

Compared with the prior art, the present disclosure at least has the following advantages:

(1) the provided micro-array may be formed by randomly dispersing the plurality of microchips on the substrate surface (namely the carrying surface), and it is not needed that each array point is distributed at a specific and preset position, so operations are simple, the costs are low, and a problem that each array point is contaminated with each other does not generate in the preparation procedure;

(2) the utilized encoded microchips are easy to be accurately processed and prepared in batch by using a micromachining technique method, so the costs are relatively lower and the batch repeatability is great; their coding characteristic structures only depend on the machining accuracy and are not influenced by the solution, the dye and the photoelectric field, so the decoding accuracy is high; meanwhile, the size of the utilized encoded microchips is small, wherein by taking a carrier of a 96-well plate for an example, the number of the encoded microchips contained in each well is up to hundreds even thousands, and tens of parallel detections may be achieved;

(3) the provided micro-array may match conventional fluorescence agents, chemiluminescence agents, bioluminescence agents and the like to achieve detections, thereby largely reducing costs of consumables; and

(4) the provided micro-array may match conventional analysis devices such as a fluorescence or chemiluminescence microscopic imaging device and the like in use, design and manufacturing techniques are mature, and operations and maintenance are easy to achieve in use.

In sum, the technical schemes of the present disclosure show outstanding advantages in the aspects of multiplicity, efficiency, sensitivity and the like of detection, high-throughput and high-precision sample detection can be achieved, operations are simple, and preparation is simple, convenient and economical.

The following describes the technical schemes in the embodiments of the present disclosure in detail with reference to some embodiments and the accompanying drawings. Apparently, the described embodiments are merely a part rather than all of the embodiments of the present disclosure. All other embodiments obtained by a person of ordinary skill in the art based on the embodiments of the present disclosure without creative efforts shall fall within the protection scope of the present disclosure.

Embodiment 1: detection on a plurality of oligonucleotide fragments in a biological sample (nucleic acid detection).

Embodiment 2: detection on a plurality of antigen proteins in a clinical sample (immunodetection)

Embodiment 3: detection on a plurality of medicinal molecules in a medicolegal sample.

In the embodiments, the utilized encoded microchips may be selected from a group including, but not limited to, fluorescent encoded micro-particles, graph encoded microchips, magnetic Luminex beads and the like.

### Embodiment 4

In the present embodiment, a micro-fluidic reaction device based on a glass slide and PDMS is manufactured.

The experimental procedure comprises the following steps.

The device comprises two components: 1) a glass substrate, the surface of which is coated with an incompletely cured PDMS film; and 2) a PDMS cover having a micro-fluidic groove. Its manufacturing steps are as follows:

1. processing a glass slide by using a PDC-MG plasma cleaning machine (which uses oxygen as the air source), so that the surface of the glass slide is fully activated;

2. placing the glass slide on a spin coater, and adding a negative-pressure suction sheet; dropwise adding a PDMS mixture, in which a ratio of a monomer to an initiator is 10:1, to the surface of the glass slide, and then running the spin coater for two minutes at the speed of 2000 rpm, so that the PDMS mixture uniformly coats the surface of the glass slide;

3. transferring the glass slide into an oven with the temperature of 80 degrees centigrade to stay for five minutes, so that the PDMS film on the surface of the glass slide is partially cured via the polymerization reaction;

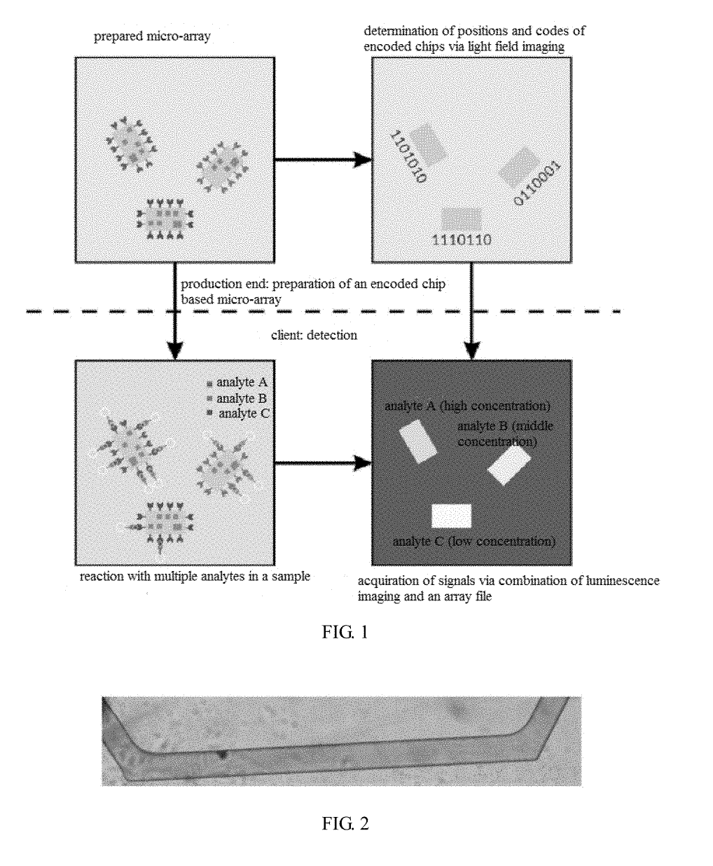

4. taking out the glass slide, and placing a PDMS cover (the surface of which has a micro-fluidic groove with the width of 1 mm and the depth of 100 microns) manufactured by the micromachining technique on the surface coated with the PDMS film, therefore, the groove and the surface of the glass slide form a closed cavity structure (namely a micro-fluidic channel), the structure of the micro-fluidic channel under the microscope refers to FIG. 2, and as shown in FIG. 2, a white rule represents 10 mm; and

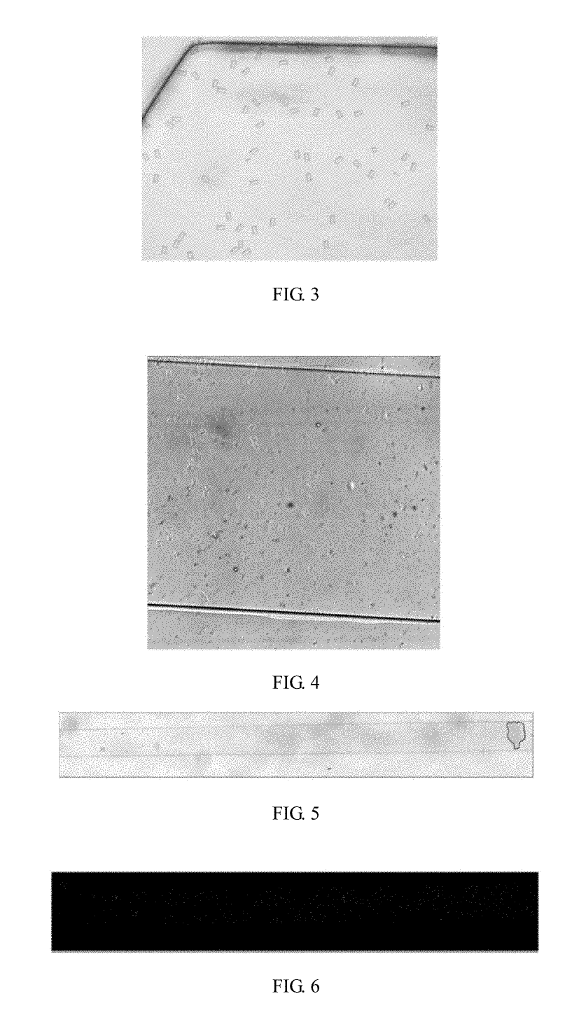

5. injecting various pre-mixed encoded microchip (the surface of which is coupled with the corresponding capture ligand such as an antibody, DNA and the like) suspension liquid into the interior of the micro-fluidic channel through the opening of the groove in the PDMS cover by using a micro-injection pump, and placing it at the temperature of four degrees centigrade for 24 hours, in this procedure, the microchips firstly deposit on the surface of the partially cured and sticky PDMS film and then are firmly combined with the surface of the film in the low-temperature placement procedure when the film is gradually and completely cured, and meanwhile, the PDMS micro-fluidic channel is also sealed, specifically referring to FIG. 3 and FIG. 4.

At this time, a micro-fluidic reaction device in which a sample solution may be injected to be detected is obtained.

### Embodiment 5

Hybridization of a PCR product is carried out in the micro-fluidic reaction device with various encoded chips in order to detect various human papillomavirus (HPV) in a detection sample.

The experimental procedure comprises the following steps:

HPV gene fragments in a sample are amplified through a multiple-amplification PCR reaction, and amplification products are added to the micro-fluidic channel in order to be hybridized with a chip, the surface of which is connected with a corresponding DNA capture probe, so that a parallel detection is carried out on each HPV subtype virus in the sample. Its main experimental steps are as follows:

1. manufacturing of a micro-fluidic reaction device, wherein amino-modified DNA capture probes (which aim at four high-risk HPVs including HPV16, HPV18, HPV18 and HPV58) are covalently linked to the surfaces of chips with different codes (the number of chips with the same code is about 200), and the chips and the micro-fluidic channel are assembled according to embodiment 4 to form the reaction device;

2. preparation of PCRmix by using TaKaRa Taq HS PCRKit, UNGplus, wherein each element is added according to the operating instruction of the kit;

| Primer Name | Sequence (5′ to 3′) | Notes |
|---|---|---|
| MY11 | GCMCAGGGWCATAAYAATGG | outer primer |
| MY09 | CGTCCMARRGGAWACTGATC | outer primer |
| Pr-[PGMY09-51] | GCGACCCAATGCAAATTGGT | outer primer |
| Pr-[PGMY11-b] | GCGCAGGGCCACAATAATGG | outer primer |
| Pr-[PGMY11-f] | GCTCAGGGACACAATAATGG | outer primer |
| Pr-[PGMY09-k]-5'Biotin | CGTCCAAGGGGATACTGATC | outer primer |
| Pr-[52_Rv] | CTACCTAAAGGAAACTGATC | outer primer |
| Pr-[GP6-43]-5′Biotin | GAAATATAAACTGCAGATCACATTC | inner primer |
| Pr-[GP6+]-5′Biotin | GAAAAATAAACTGTAAATCATATTC | inner primer |
| Pr-[GP6-de]-5′Biotin | GAAAHAYAAAYTGYAADTCAWAYTC | inner primer |
| Pr-[GH2O] | GAAGAGCCAAGGACAGGTAC | outer primer |
| Pr-[PCO4]-5′Biotin | CAACTTCATCCACGTTCACC | outer primer |
| Pr-[β_Rv-inner]-5′Biotin | GGCAGACTTCTCCTCA | inner primer |

3. PCR amplification, wherein a single-strand DNA PCR product is obtained through a nest-asymmetric PCR amplification target sequence; totally two cycles of amplification need to be carried out, wherein the first amplification cycle uses the outer primer, and the second amplification cycle uses the inner primer; fluorescence or biotin is labeled on a reverse primer; in the first PCR cycle, a higher annealing temperature is utilized to carry out amplification, and the outer primer plays the leading role; in the second PCR cycle, a lower annealing temperature is utilized, most of the outer primer is consumed, and the inner primer plays the leading role, so that a short single-strand DNA product is generated. An ABI9500PCR device runs the following program:

| | | | | |
|---|---|---|---|---|
| 95° C. | 2 | min | | |
| 94° C. | 20 | s | | |
| 55° C | 30 | s | {close oversize brace} | 15 cycles |
| 72° C. | 1 | min | | |
| 94° C. | 20 | s | | |
| 50° C. | 30 | s | {close oversize brace} | 30 cycles |
| 72° C. | 1 | min | | |
| 72° C. | 5 | min | | |

4. hybridization, wherein a hybridization system (50 microlitres) is prepared by adding hybridization buffer solution (5*SSC, 0.05% Tween20) to the PCR product of 10 microlitres till the solution mixture is up to 50 microlitres, the hybridization system is injected into the micro-fluidic reaction device through an injection pump, and the device undergoes water bath for five minutes at the temperature of 95 degrees centigrade and ice bath for one minute, is cultured for two hours at the temperature of 55 degrees centigrade and then is washed twice by using a cleaning solution of 200 microlitres (1*SSC, 0.01% Tween20);

5. detection, wherein streptavidin-phycoerythrin (SAPE) of 2 micrograms per milliliter is injected to be cultured (to generate a reaction with the biotin on the captured product DNA) for one hour and is washed twice by using the cleaning solution of 200 microlitres; a Nikon Ti-U reversed fluorescence microscope is used for carrying out imaging, the codes of chips are identified by a light field, and target signals are analyzed by fluorescence; light field and fluorescence images of the micro-fluidic channel after the reaction respectively refer to FIG. 5 and FIG. 6; and

6. chip and a corresponding target combination specificity results refer to the following table, wherein it can be seen that combination signals of chips with capture probes and corresponding targets are remarkably higher than signals generated by non-corresponding targets.

| | Chip No. | | | | | |
|---|---|---|---|---|---|---|
| | #093 | #111 | #033 | #127 | #60 | #90 |
| Target | HPV16 | HPV18 | HPV33 | HPV35 | HPV39 | β-globulin |
| HPV16 | 5935.58 | 199.83 | 342.12 | 717.21 | 159.73 | 569.70 |
| HPV18 | 266.75 | 8353.24 | 225.38 | 680.99 | 65.29 | 344.31 |
| HPV33 | 8.55 | 48.20 | 5316.02 | 716.00 | 80.00 | 328.17 |
| HPV35 | 79.13 | 83.71 | 286.94 | 5026.66 | 140.13 | 117.73 |
| HPV39 | 105.48 | 285.41 | 397.90 | 796.93 | 3095.51 | 210.56 |
| β-globulin | 111.54 | 111.24 | 238.46 | 649.38 | 147.15 | 6406.53 |
| Negative | 205.55 | 140.25 | 142.63 | 625.51 | 131.79 | 239.63 |
| Control | | | | | | |

It should be understood that the foregoing merely is specific embodiments of the present disclosure. It should be pointed out that a plurality of improvements and modifications made by a person of ordinary skill in the art without departing from the principle of the present disclosure shall fall within the protection scope of the present disclosure.

1. (canceled)

2. A preparation method of encoded chip based micro-array, comprising:
providing a carrier, wherein the carrier has at least one fluid accommodating cavity, and at least one carrying surface is distributed in the fluid accommodating cavity, and the carrying surface is a curved surface or a flat surface, preferably the flat surface;
uniformly coating the carrying surface with an adhesive;
adding a selected fluid to the fluid accommodating cavity till the carrying surface is immersed by the selected fluid;
depositing encoded microchips dispersed in the selected fluid on the carrying surface, and enabling the encoded microchips to be combined with the adhesive distributed on the carrying surface; and
curing the adhesive, thereby fixing the encoded microchips onto the carrying surface.

3. The preparation method according to claim 2, comprising:
uniformly dispersing the plurality of encoded microchips in a solvent, thereby forming an encoded microchip suspension liquid to be as the selected fluid, wherein the solvent comprises water and/or an organic solvent, preferably the solvent is selected from water or a buffer solution;
then adding the encoded microchip suspension liquid to the fluid accommodating cavity till the carrying surface is immersed by the encoded microchip suspension liquid;
depositing the plurality of encoded microchips in the encoded microchip suspension liquid on the carrying surface in a discrete state, and enabling the encoded microchips to be combined with the adhesive distributed on the carrying surface; and
curing the adhesive, thereby fixing the plurality of encoded microchips onto the carrying surface and then forming an encoded microchip based micro-array.

4. The preparation method according to claim 2, comprising:
adding the selected fluid to the fluid accommodating cavity till the carrying surface is immersed by the selected fluid, wherein the selected fluid comprises water and/or an organic solvent, preferably the solvent is selected from water or a buffer solution;
dispersing the plurality of encoded microchips into the selected fluid, depositing the plurality of encoded microchips on the carrying surface in a discrete state, and enabling the encoded microchips to be combined with the adhesive distributed on the carrying surface; and
curing the adhesive, thereby fixing the plurality of encoded microchips onto the carrying surface and then forming an encoded microchip based micro-array.

5. The preparation method according to claim 2, comprising: depositing the encoded microchips dispersed in the selected fluid on the carrying surface under the action of any one or at least two components selected from a group of a gravity field, an external magnetic field and an external electric field, and enabling the encoded microchips to be combined with the adhesive distributed on the carrying surface.

6. The preparation method according to claim 2, comprising: depositing the encoded microchips dispersed in the selected fluid on the carrying surface under their own weights, and enabling the encoded microchips to be combined with the adhesive distributed on the carrying surface, wherein the density of the encoded microchips is greater than the density of the selected fluid.

7. (canceled)

8. The preparation method according to claim 2, wherein the encoded microchips are distributed on the carrying surface in a lying state, while the carrying surface is a horizontal carrying surface; and/or, the carrying surface is the bottom end surface of the fluid accommodating cavity; and/or, the carrier comprises a multi-well plate, a micro-plate, a micro-fluid based device or a container similar to a watch glass; and/or, the size of the encoded microchips is from 1 micron to 100 microns.

9. (canceled)

10. The preparation method according to claim 2, comprising: applying a fluidic adhesive onto the carrying surface and ensuring that the adhesive naturally spreads out to form a plane on the carrying surface, or uniformly coating the carrying surface with the fluidic adhesive.

11-13. (canceled)

14. The preparation method according to claim 2, wherein the encoded microchips comprise substrates with optical identification codes, or the encoded microchips comprise substrates and microstructures that are connected with the substrates and are used as the optical identification codes, wherein the optical identification codes comprise graph identification codes or spectrum codes, preferably the optical identification codes are selected from the graph identification codes.

15. (canceled)

16. The preparation method according to claim 2, wherein the encoded microchips comprise transparent substrates and opaque planar microstructures as the graph identification codes, wherein the opaque planar microstructures are distributed on the surfaces of the transparent substrates.

17. The preparation method according to claim 2, wherein the encoded microchips are selected from silicon-based encoded microchips, and the adhesive is selected from silicone adhesives.

18. The preparation method according to claim 2, wherein the substrate is further fixed with a capture substance capable of specifically capturing a target substance.

19. (canceled)

20. The preparation method according to claim 2, further comprising: imaging and identifying the plurality of encoded microchips distributed on the carrying surface, thereby locating and decoding each encoded microchip and obtaining a dataset comprising locating information and decoding information corresponding to the plurality of encoding microchips distributed on the carrying surface.

21. (canceled)

22. An encoded chip based micro-array comprising: a carrier and a plurality of encoded microchips, wherein the carrier has at least one fluid accommodating cavity, at least one carrying surface is distributed in the fluid accommodating cavity, and the plurality of encoded microchips are distributed on the carrying surface in a discrete state and are adhered and fixed to the carrying surface, wherein the carrying surface is a flat surface or a curved surface, preferably the flat surface.

23. The encoded chip based micro-array according to claim 22, wherein the encoded microchips are fixed to and combined with the carrying surface through the cured adhesive coating the carrying surface.

24. (canceled)

25. The encoded chip based micro-array according to claim 22, wherein the encoded microchips are distributed on the carrying surface in a lying state, while the carrying surface is a horizontal carrying surface.

26. (canceled)

27. The encoded chip based micro-array according to claim 22, wherein the carrying surface is the bottom end surface of the fluid accommodating cavity; and/or, the carrier comprises a multi-well plate, a micro-plate, a micro-fluid based device or a container similar to a watch glass; and/or, the size of the encoded microchips is from 1 micron to 100 microns.

28. (canceled)

29. The encoded chip based micro-array according to claim 22, wherein the encoded microchips comprise substrates with optical identification codes; or the encoded microchips comprise substrates and microstructures that are connected with the substrates and are used as the optical identification codes; and the optical identification codes comprise graph identification codes or spectrum codes, preferably the optical identification codes are selected from the graph identification codes.

30. The encoded chip based micro-array according to claim 29, wherein the encoded microchips comprise transparent substrates and opaque planar microstructures as the graph identification codes, wherein the opaque planar microstructures are distributed on the surfaces of the transparent substrates.

31. The encoded chip based micro-array according to claim 22, wherein the encoded microchips are selected from silicon-based encoded microchips, and the adhesive is selected from silicone adhesives.

32. The encoded chip based micro-array according to claim 22, wherein the substrate is further fixed with a capture substance capable of specifically capturing a target substance.

33. (canceled)

34. A biochemical kit comprising:
the encoded chip based micro-array according to claim 22,
a dataset, wherein the dataset comprises locating information and decoding information corresponding to the plurality of encoded microchips distributed on the carrying surface,
an operating instruction, and.
at least one of a buffering solution, a detection agent, diluent and lotion, wherein the detection agent comprises at least one component selected from a group of a detection antibody or ligand with a fluorescent dye label, a detection antibody or ligand with a biotin label, an avidin-fluorescent protein conjugate, an avidin-horseradish peroxidase compound and a chemiluminiscence agent.

35-38. (canceled)

39. A biochemical detection system, comprising:
the biochemical kit according to claim 34;
an optical imaging device, which at least is used for collecting associated image information when the biochemical kit detects a to-be-detected analyte, wherein the image information collected by the optical imaging device comprises at least one of image coding information, optical strength information and spectrum coding information, which are shown by each encoded microchip after each encoded microchip and the to-be-detected analyte generate reactions; and
a data processing device, which at least is used for processing the image information collected by the optical imaging device and combining the dataset in the biochemical kit to achieve qualitative and/or quantitative detection on the to-be-detected analyte.

40-41. (canceled)

42. A biochemical detection method, comprising:
providing the biochemical kit according to claim 34;
adding a liquid phase sample possibly including the target substance to the fluid accommodating cavity of the micro-array, and ensuring that the target substance is combined with the capture substance fixed to the carrier; and
by an imaging device, collecting image information of the micro-array, and comparing the collected image information with the dataset in the biochemical kit so as to achieve qualitative and/or quantitative detection on the to-be-detected analyte, wherein the image information collected by the optical imaging device comprises at least one of image coding information, optical strength information and spectrum coding information, which are shown by each encoded microchip after each encoded microchip and the to-be-detected analyte generate reactions.

43. (canceled)
